(19) United States
(12) Patent Application Publication
(10) Pub. No.: US 2008/0271732 A1
(43) Pub. Date: Nov. 6, 2008

(54) Pulmonary Drug Delivery Devices Configured to Control the Size of Administered Droplets

(75) Inventors: Philip Weaver, Mouth of Wilson, VA (US); Charles Eric Hunter, Jefferson, NC (US); Lyndell Duvall, Fleetwood, NC (US); Jack Hebrank, Durham, NC (US); George Colvard, Crumpler, NC (US); Timothy Roland, W. Jefferson, NC (US); Andy Pierce, Fleetwood, NC (US)

Correspondence Address: THOMAS, KAYDEN, HORSTEMEYER & RISLEY, LLP, 600 GALLERIA PARKWAY, S.E., STE 1500, ATLANTA, GA, 30339-5994, US

(73) Assignee: NEXT SAFETY, INC., Jefferson, NC (US)

(21) Appl. No.: 12/037,513

(22) Filed: Feb. 26, 2008

Related U.S. Application Data
(63) Continuation-in-part of application No. 11/950,180, filed on Dec. 4, 2007.
(60) Provisional application No. 60/915,379, filed on May 1, 2007.
(60) Provisional application No. 60/915,390, filed on May 1, 2007.
(60) Provisional application No. 60/915,408, filed on May 1, 2007.

(51) Int. Cl. A61M 16/10; A61M 11/00

(52) U.S. Cl. 128/200.14; 128/200.16; 128/204.17

(57) ABSTRACT

A pulmonary drug delivery device including a drug delivery tube that defines a flow path and a droplet ejection device configured to eject droplets of medication into the flow path. Using collected feedback, the pulmonary drug delivery device can control the size of the droplets that are administered to a user.

### CROSS-REFERENCES TO RELATED APPLICATIONS

This application claims priority to U.S. provisional application Ser. No. 60/915,379 entitled “Controlling the Droplet Size in a Drug Delivery System Using Temperature Modification” and filed May 1, 2007, U.S. provisional application Ser. No. 60/915,390 entitled “Aerosol Generating Device” and filed May 1, 2007, and U.S. provisional application Ser. No. 60/915,408 entitled “Droplet Delivery Methods and Systems” and filed May 1, 2007. This application also comprises a continuation-in-part of U.S. non-provisional application Ser. No. 11/950,180 entitled “Systems, Methods, and Apparatuses for Pulmonary Drug Delivery” and filed on Dec. 4, 2007, and U.S. non-provisional application Ser. No. 11/950,154 entitled “Apparatuses and Methods for Pulmonary Drug Delivery” and filed on Dec. 4, 2007. Each of the foregoing applications is hereby entirely incorporated by reference into the present disclosure.

### BACKGROUND

The lung is the essential respiration organ in air-breathing vertebrates, including humans. Its principal function is to transport oxygen from the atmosphere into the bloodstream, and to excrete carbon dioxide from the bloodstream into the atmosphere. This exchange of gases is accomplished by a mosaic of specialized cells that form millions of tiny, thin-walled air sacs called alveoli. Beyond respiratory functions, the lungs also act as an efficient drug delivery mechanism.

In recognition of the potential of pulmonary drug delivery, various efforts have been made toward developing effective pulmonary drug delivery devices. Current pulmonary drug delivery devices include metered dose inhalers (MDIs), dry powder inhalers (DPIs), and nebulizers. MDIs are pressurized hand-held devices that use propellants for delivering liquid medicines to the lungs. DPIs also use propellants, but deliver medicines in powder form. Nebulizers, also called “atomizers,” pump air or oxygen through a liquid medicine to create a vapor that is inhaled by the patient.

Each of the above-described devices suffer from disadvantages that decrease their attractiveness as a mechanism for pulmonary drug delivery. For example, when MDIs are used, medicine may be deposited at different levels of the pulmonary tree, and therefore may be absorbed to different degrees, depending on the timing of the delivery of the medicine in relation to the inhalation cycle. Accordingly, actual deposition of medicine in the lungs during patient use may differ from that measured in a controlled laboratory setting. Furthermore, a portion of the “metered dose” may be lost in the mouthpiece or the oropharynx.

Although DPIs reflect an effort to improve upon MDIs, small volume powder metering is not as precise as the metering of liquids. Therefore, the desired dosage of medicine may not actually be administered when a DPI is used. Furthermore, ambient environmental conditions, especially humidity, can adversely effect the likelihood of the medicine actually reaching the lungs.

Nebulizers may also exhibit unacceptable variability in delivered dosages, especially when they are of the inexpensive, imprecise variety that is common today. Although more expensive nebulizers are capable of delivering more precise dosages, the need for a compressed gas supply that significantly limits portability and the need for frequent cleaning to prevent bacterial colonization renders such nebulizers less desirable. Furthermore, the relatively high cost of such nebulizers also makes their use less attractive.

From the above, it can be appreciated that it would be desirable to have an improved pulmonary drug delivery system or device that avoids one or more of the above-described disadvantages.

Of the various applications in which such an improved pulmonary drug delivery system and device could be used, the delivery of nicotine as a method to achieve smoking cessation is one of the most compelling. The adverse health care consequences of smoking tobacco are enormous and incontrovertible. According the World Health Organization (WHO), tobacco is the second major cause of death in the world, currently accounting for one in ten deaths worldwide (5 million each year), and is the single largest preventable cause of disease and premature death. Of the 1.1 billion smokers in the world today, half will die from tobacco-related illness. For example, it is estimated that smoking will contribute to the death of one third of all Chinese males under 30 years old currently alive. In the United States, the 1999 National Health Interview Survey estimated that 46.5 million adults smoke and that 440,000 die each year from smoking related causes. In men, smoking is estimated to decrease life expectancy by 13.2 years and in women by 14.5 years.

Furthermore, it is now understood that cigarette smoke is not only harmful to the smoker, but also can affect the health of non-smokers when they passively inhale the smoke of other peoples' cigarettes. Such “secondhand smoke” is a risk factor for numerous types of adult ailments including lung cancer, breast cancer, and heart disease. Secondhand smoke exposure also increases the risk of various diseases in children and infants.

Despite recognizing the health risks associated with their habit, smokers continue to smoke. The primary reason for this phenomenon relates to the effect that nicotine has on the central nervous system (CNS). At low serum levels, nicotine provides stimulatory effects, primarily through activation of the locus ceruleus within the cerebral cortex. Such stimulatory effects include increased concentration, decreased anxiety, improved mood, decreased appetite, and improved memory. At high serum levels, nicotine activates the limbic system and produces a sense of euphoria, commonly referred to as a “buzz.” Cigarette smokers are accustomed to achieving both of these effects.

After inhaling cigarette smoke, nicotine is absorbed across the alveolar membrane in the lungs, leading to a rapid rise of serum nicotine levels within a few seconds. Within five minutes of smoking, the average maximum concentration of nicotine in arterial blood rises to 49 nanograms per milliliter (ng/ml), thereby providing the euphoric buzz. As nicotine levels fall, the stimulant effects predominate for the next 1-2 hours. Soon after, however, withdrawal symptoms begin to develop. These symptoms include irritability, anger, impatience, restlessness, difficulty concentrating, increased appetite, anxiety, and depressed mood. Such withdrawal symptoms are normally relieved by smoking the next cigarette, thereby creating a potentially endless cycle.

Over the years, many efforts have been made to develop effective means for assisting smokers in quitting. Currently, there are several Federal Drug Administration (FDA) approved nicotine replacement treatments (NRTs) intended for use in smoking cessation available both over-the-counter and as a prescription. Significantly, none of those NRTs deliver significant amounts of nicotine to the alveolar level of the lungs. Instead, they rely on the absorption of nicotine across the skin or across the nasal, buccal, or oropharyngeal mucosa. As a result, absorption is much slower and much less efficient than that typical of smoking and therefore leads to slower and much lower peak nicotine concentrations compared to that produced by cigarettes. Notably, this is true for existing nicotine inhalers, which are purported to have delivery characteristics most like cigarettes. Studies have confirmed that nicotine absorption resulting from use of such inhalers primarily occurs across the buccal mucosa, not the lungs, and that the arterial nicotine concentration spike that results from cigarette smoking does not occur with such inhalers.

The peak serum levels achieved with the current NRTs may be adequate to ameliorate or prevent withdrawal symptoms. However, they do little to satisfy the acute craving for the “buzz” created by the rapid onset and high peak serum nicotine levels typical of tobacco smoke. This may be the primary reason why so few habitual smokers that have used NRT have achieved long-term success. Instead, such persons typically give in to the persistent cravings, which currently can only be satisfied through smoking.

Given the enormity of the health problems caused by smoking, it is agreed upon by physicians and laypersons alike that the best thing that smokers can do is quit smoking. However, given the limited success that previous cessation solutions have had, it is clear that more effective alternatives are needed. It stands to reason that an alternative capable of providing nicotine to the user in ways analogous to smoking could save numerous lives.

### BRIEF DESCRIPTION OF THE DRAWINGS

The disclosed pulmonary drug delivery devices can be better understood with reference to the following drawings. The components in the drawings are not necessarily to scale.

FIG. 1 is front perspective view of an embodiment of a device for pulmonary drug delivery.

FIG. 2 is a rear perspective view of the device of FIG. 1.

FIG. 3 is a front perspective view of the device of FIG. 1 with a front cover of the device removed.

FIG. 4 is an exploded perspective view of the device of FIG. 1.

FIG. 5 is side perspective view of a drug delivery member of the device of FIG. 1.

FIG. 6 is a cross-sectional side view of the drug delivery member of FIG. 5.

FIG. 7 is a side perspective view of a medicine storage and delivery unit of the drug delivery member of FIG. 5.

FIG. 8 is a first rear perspective view of the drug delivery member of FIG. 5, shown with an electrical conductor decoupled from the member.

FIG. 9 is a second rear perspective view of the drug delivery member of FIG. 5, shown with the electrical conductor coupled to the member.

FIG. 10 is bottom perspective view of the medicine storage and delivery unit shown of FIG. 7, illustrating a droplet ejection device of the unit.

FIG. 11 is a side view of an alternative medicine storage and delivery unit including a first heating element.

FIG. 12 is bottom perspective view of an alternative medicine storage and delivery unit including a second heating element.

FIG. 13 is timing diagram that illustrates the timing for pulses that are applied to ejection elements.

FIG. 14 is a side view of an alternative medicine storage and delivery unit including two separate compartments and a mixing chamber.

FIG. 15 is bottom perspective view of an alternative medicine storage and delivery unit including multiple rows of ejection nozzles having different sizes.

FIG. 16 is a side view of an alternative drug delivery member including a heating element.

FIG. 17 is a side view of a first alternative support structure including an electromagnetic energy source.

FIG. 18 is a side view of a second alternative support structure including an electromagnetic energy source.

FIG. 19 is a side view of an alternative drug delivery member including a conditioning unit.

FIG. 20 is a side view of an alternative drug delivery member including a sonic wave generator.

FIG. 21 is a top view of an alternative embodiment of a drug delivery member that can be used in the device of FIG. 1.

FIG. 22 is an end view of the drug delivery member of FIG. 21.

FIG. 23 is a schematic view of an apparatus for sensing the size of ejected droplets.

### DETAILED DESCRIPTION

As described above, it would be desirable have a pulmonary drug delivery system or device that is effective in enabling absorption of medicines, such as nicotine, via the lungs. Embodiments of a pulmonary drug delivery device are described in the following disclosure.

Disclosed herein are various embodiments of apparatuses and methods for pulmonary drug delivery. It is noted that those embodiments comprise mere implementations of the disclosed inventions and that alternative embodiments are both possible and intended to fall within the scope of the present disclosure.

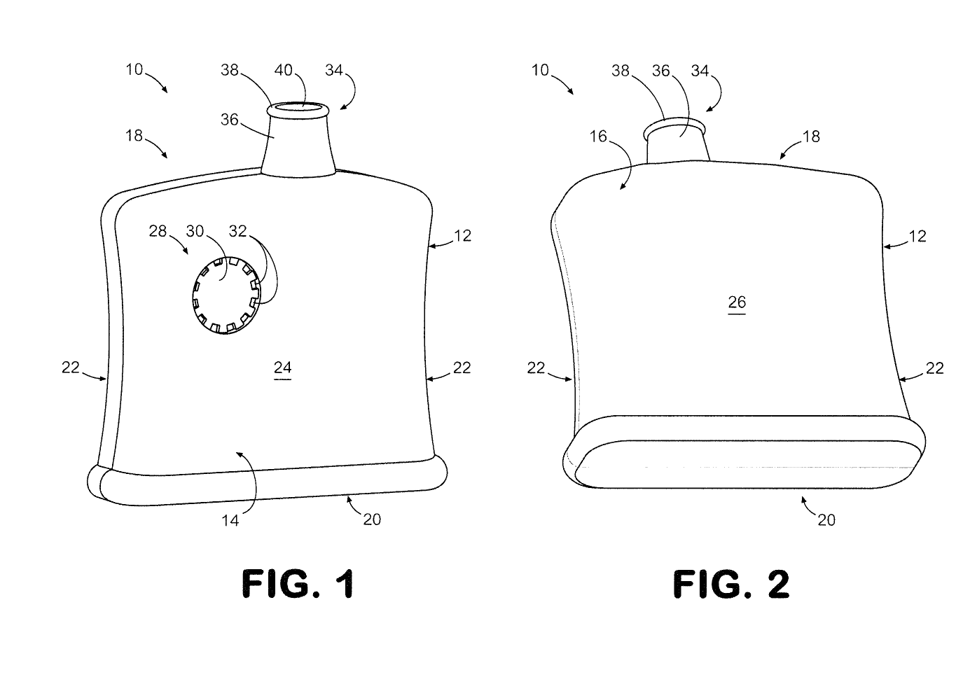

Referring to the drawings, in which like numerals indicate corresponding parts throughout the several views, FIGS. 1 and 2 illustrate an example pulmonary drug delivery device 10. In at least some embodiments, the device 10 comprises a portable (e.g., handheld) device that can be easily carried with the user throughout the day so as to be available whenever needed. The device 10 includes an outer housing 12 that generally comprises a front side 14, a rear side 16, a top side 18, a bottom side 20, and opposed lateral sides 22. In some embodiments, the device 10 comprises a front cover 24 that defines the front side 14 and portions of the top side 18, bottom side 20, and opposed lateral sides 22, and a rear cover 26 that defines the rear side 16 and portions of the top side 18, bottom side 20, and opposed lateral sides 22. Provided on the front side 14, for example formed within the front cover 24, is an air inlet 28 that enables ambient air to flow from the environment in which the device 10 is used into an interior space of the device. In the illustrated embodiment, the inlet 28 comprises a generally circular depression 30 that includes a plurality of openings or slots 32 that are formed around the periphery of the depression.

Extending from the top side 18 of the housing 12 is a mouthpiece 34 that is used to deliver medicine to a patient who uses the device 10 (i.e., a “user”). In the embodiment of FIGS. 1 and 2, the mouthpiece 34 comprises a hollow, frustoconical member 36 that terminates in a rounded lip 38 that the user can place in his or her mouth or between his or her lips. As is apparent from FIG. 1, the mouthpiece 34 includes an opening 40 at its end that serves as an outlet for the device 10.

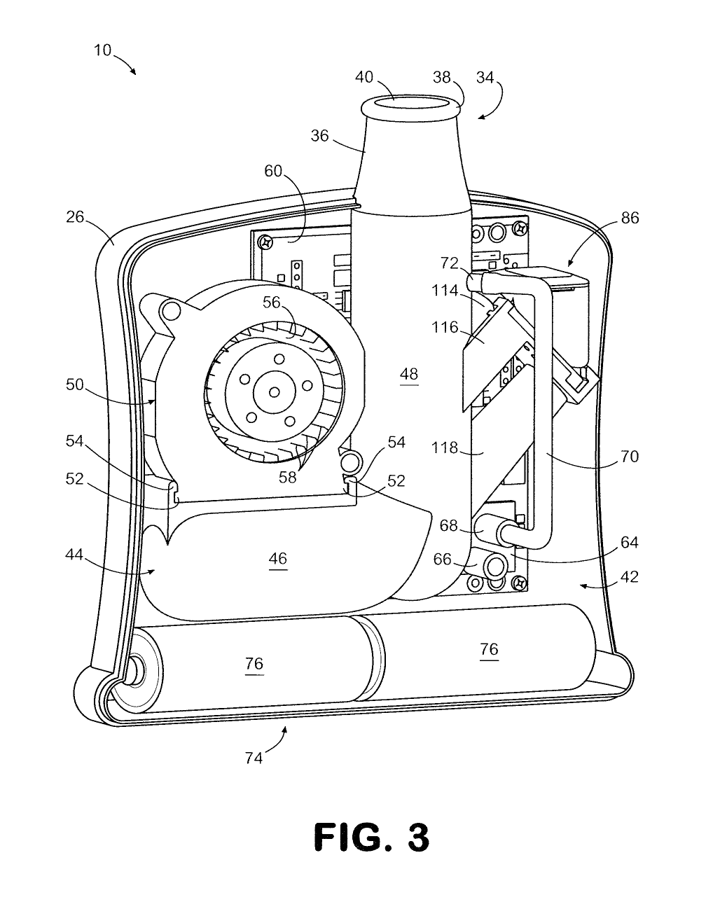

FIG. 3 is a front perspective view of the device 10 with the front cover 24 removed to reveal the interior space 42 defined by the device and, more particularly, by its two covers 24, 26. As indicated in FIG. 3, provided within the interior space 42 is a drug delivery member 44. As is also indicated in FIG. 3, the drug delivery member 44 is generally L-shaped and therefore comprises a first or lower tube 46 that is in fluid communication with a second or upper tube 48. In the illustrated embodiment, the lower tube 46 is horizontally arranged and the upper tube 48 is vertically arranged such that the bottom and upper tubes form a sharp angle between them. As described in greater detail below, that sharp angle both facilitates suspension of ejected droplets of medicine within air that flows through the drug delivery member 44 and desired evaporation of the droplets before they exit the drug delivery member. In some embodiments, the bottom and upper tubes 46, 48 are unitarily formed together, for example using an injection molding process. Likewise, the mouthpiece 34 can be unitarily formed with the upper tube 48 such that each of the lower tube 46, upper tube 48, and mouthpiece 34 are formed from a single, continuous piece of material, such as a plastic material. Given that they form a continuous tube in such an arrangement, the lower tube 46, upper tube 48, and mouthpiece 34 can be generally considered to form a drug delivery tube.

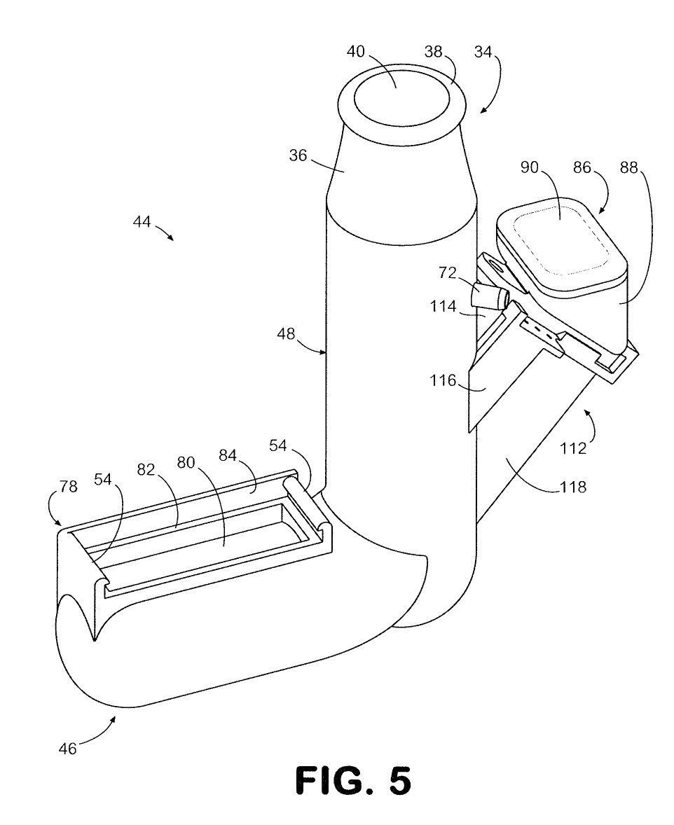

Also provided within the interior space 42 is a fan 50 that is used to generate airflow within the drug delivery member 44. As indicated in FIG. 3, the fan 50 can, in some embodiments, mount to the drug delivery member 44, for example to the lower tube 46. In such embodiments, the fan 50 can comprise mounting flanges or lugs 52 that are retained by brackets 54 provided on the lower tube 46 (see FIG. 5). Irrespective of how or whether the fan 50 is mounted to the drug delivery member 44, the fan comprises an inlet 56 through which air is drawn into the fan and an outlet (not shown) from which the drawn air is exhausted from the fan at an increased velocity into the drug delivery member. In some embodiments, the fan 50 comprises a centrifugal blower that includes an internal impeller having blades 58 that force air out from the fan in direction perpendicular to the inlet 56. By way of example, the fan 50 comprises a pulse-width-modulated (PWM) centrifugal blower that outputs approximately 40 to 160 standard liters per minute (slm).

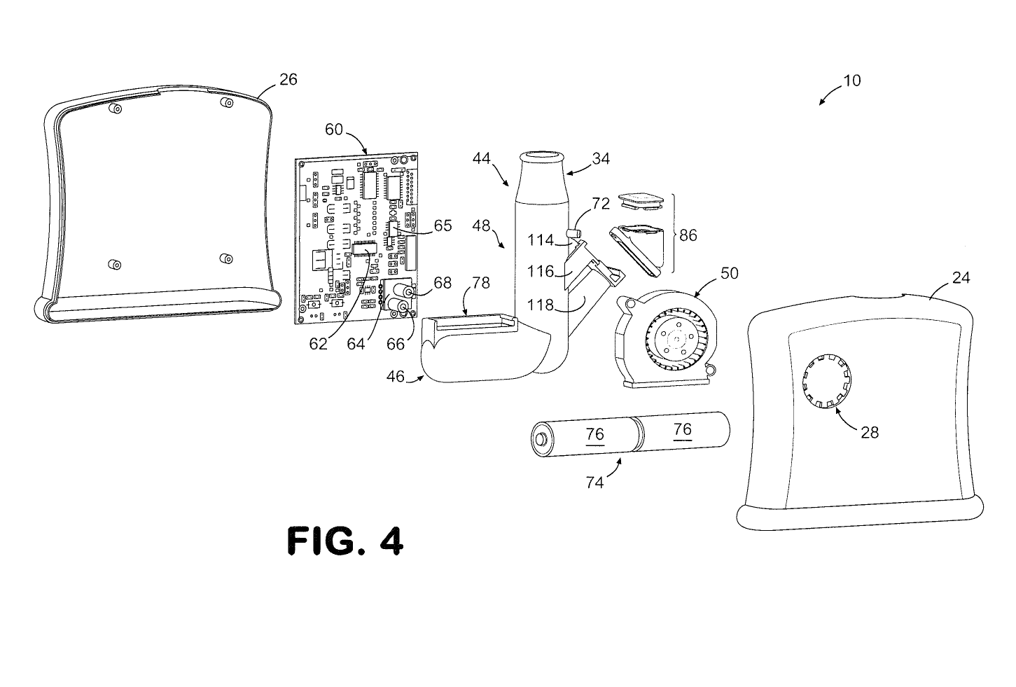

Further provided within the interior space 42 is a circuit board 60, which is more clearly shown in the exploded view of FIG. 4. The circuit board 60 generally comprises the logic that controls operation of the pulmonary drug delivery device 10. That logic can, for example, comprise a controller 62, such as a microcontroller or other processing means, that controls the operation of various components of the device 10, including the fan 50 and a droplet ejection device used to inject medicine into the airflow created by the fan. Also provided on the circuit board 60 is a pressure sensor 64, such as an integrated circuit (IC) differential pressure sensor. The pressure sensor 64 is connected to two ports: an atmospheric port 66 that is in fluid communication with the environment via the inlet 28, and an airflow port 68 that can be placed in fluid communication with the interior of the drug delivery member 44 using a coupling tube, such as tube 70 identified in FIG. 3. As indicated in FIG. 3, the tube 70 can extend from the port 68 to a further port 72 provided on (e.g., unitarily formed with) the upper tube 48 of the drug delivery member 44. With such an arrangement, the pressure sensor 64 can detect pressure drops within the upper tube 48 that are indicative of a user drawing in air from the mouthpiece 34 (i.e., inhaling from the mouthpiece). Additionally, the circuit board 60 can comprise one or more environmental condition sensors, such as sensor 65, that can be used to measure one or more of a atmospheric temperature, humidity, and pressure. As described below, such information can be used to determine what measures, if any, should be taken to control the size of droplets of medicine administered to the user.

With continued reference to FIGS. 3 and 4, the interior space 42 further includes an internal power supply 74. In some embodiments, the power supply 74 comprises alkaline batteries 76. By way of example, the batteries 76 comprise two “AA” type batteries. Irrespective of what particular type of power supply is used, the power supply 74 provides power to the fan 50, the circuit board 60, and the above-mentioned droplet ejection device. Such power can be provided with various electrical conductors (not shown) that extend from the power supply 74.

FIG. 5 illustrates the drug delivery member 44 in greater detail. As described above, the drug delivery member 44 includes a first or lower tube 46 in fluid communication with a second or upper tube 48, which is in fluid communication with a mouthpiece 34 that forms an opening 40 that functions as an outlet of the drug delivery member 44. As indicated in FIG. 5, the lower tube 46 comprises a fan mount 78 that includes a further opening 80 that functions as an inlet of the drug delivery member 44. As mentioned above, the fan mount 78 includes brackets 54 that are adapted to retain flanges or lugs 52 of the fan 50. In addition, the fan mount 78 comprises a support surface 82 that mates with and supports the fan 50 adjacent the fan's outlet, and a rear wall 84 that limits the depth of insertion of the fan relative to the fan mount 78. With such an arrangement, the fan 50 can be placed in the operating position shown in FIG. 3 by first positioning the fan lugs 52 inside of (e.g., below) the brackets 54 of the fan mount 78 and then sliding the fan along the support surface 82 until the fan abuts the rear wall 84. Once the fan 50 is placed in that position, the fan's outlet is directly adjacent the opening 80 such that air exhausted by the fan is blown directly into the lower tube 46. As described below, the air is blown in a direction that, at least in some embodiments, forms an acute angle with a longitudinal axis of the lower tube 46.

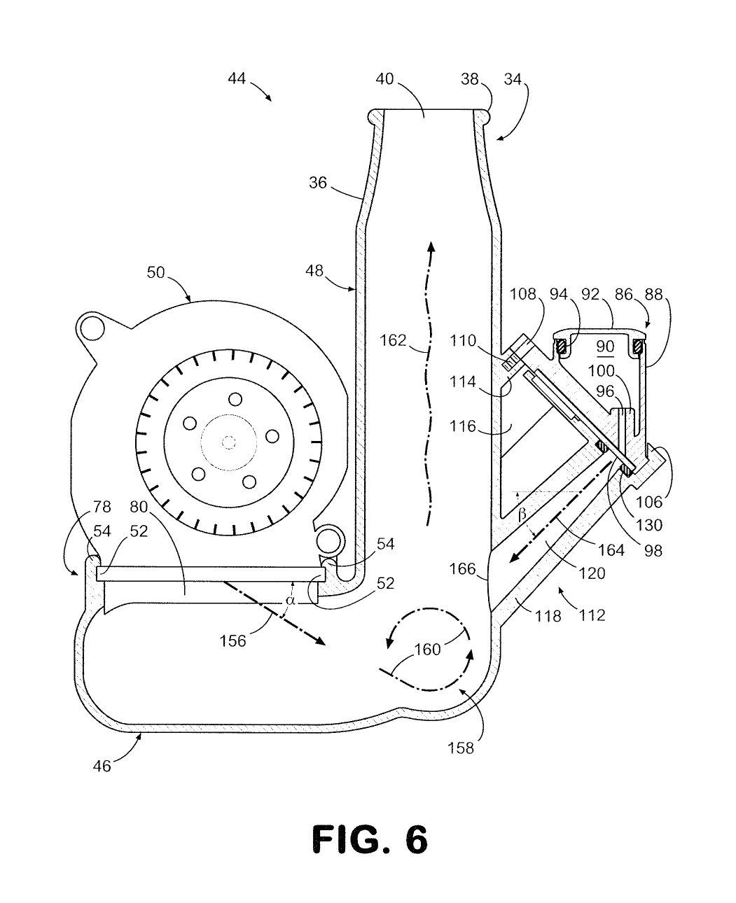

As is further indicated in FIG. 5, the drug delivery member 44 also comprises a medicine storage and delivery unit 86 that, in the illustrated embodiment, is supported by a support structure that extends from the upper tube 48. The medicine storage and delivery unit 86 comprises a medicine container 88 that, as indicated in the cross-sectional view of FIG. 6, defines an interior space 90 that can be partially or wholly filled with medicine intended for delivery to the respiratory system of the device user. In some embodiments, the medicine can be prepared immediately prior to use. For example, freebased nicotine (C₁₀H₁₄N₂) and water can be mixed together and then provided in the container 88. In some cases, the medicine can be mixed within an ampule or other independent container that is inserted into the container 88. A removable cap 92 is provided on the container 88 to reduce or prevent leakage and/or evaporation of the medicine provided within the container. In some embodiments, the cap 92 comprises a sealing member 94, such as an O-ring, that forms an airtight seal between the cap and the container 88. With further reference to FIG. 6, a passage 96 extends from the interior space 90 of the container 88 to a droplet ejection device 98, that is integrated into the medicine storage and delivery unit 86. As shown in FIG. 6, the passage 96 can be formed in a boss 100 that extends upwardly into the interior space 90.

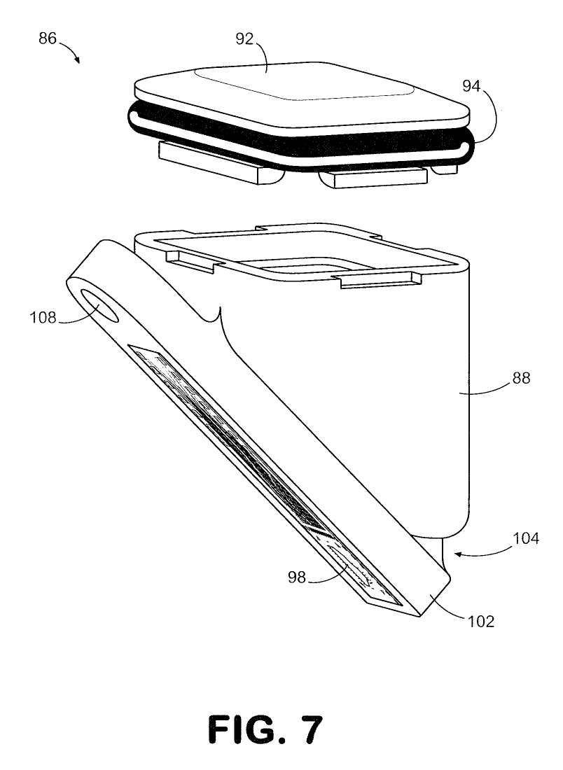

Turning to FIG. 7, the medicine storage and delivery unit 86 is provided with a support member 102 that facilitates mounting of the unit to the above-mentioned support structure. A lip 104 is formed by the support member 102 adjacent its bottom end that is adapted to be secured by a retainer clip 106 that is formed on the support structure (FIG. 6). Adjacent the top end of the support member 102 is an opening 108 that is adapted to receive a fastening element, such as a screw, that can be passed through the support member and threaded into a further opening 110 provided in the support structure (FIG. 6). Accordingly, the medicine storage and delivery unit 86 can be mounted to the support structure by first positioning the bottom lip 104 underneath the retainer clip 106 and then securing the top end of the unit to the support structure using the fastening element (not shown).

The above-mentioned support structure will now be described with reference to FIGS. 5, 6, and 8. The support structure is generally identified in FIGS. 5 and 6 by reference numeral 112. In the illustrated embodiment, the support structure 112 comprises a boss 114, two laterally opposed struts 116, and a medicine injection tube 118, each of which extends at an upward diagonal angle from the upper tube 48. The boss 114 defines the opening 110 that receives the fastening element used to secure the medicine storage and delivery unit 86 to the support structure 112. The struts 116 act as posts that provide lateral support and stability for the medicine storage and delivery unit 86. The medicine injection tube 118 also provides support and stability for the medicine storage and delivery unit 86, and further defines a pathway 120 (FIG. 6) for droplets ejected from the droplet ejection device 98 to travel before reaching a flow path defined by the bottom and upper tubes 46, 48.

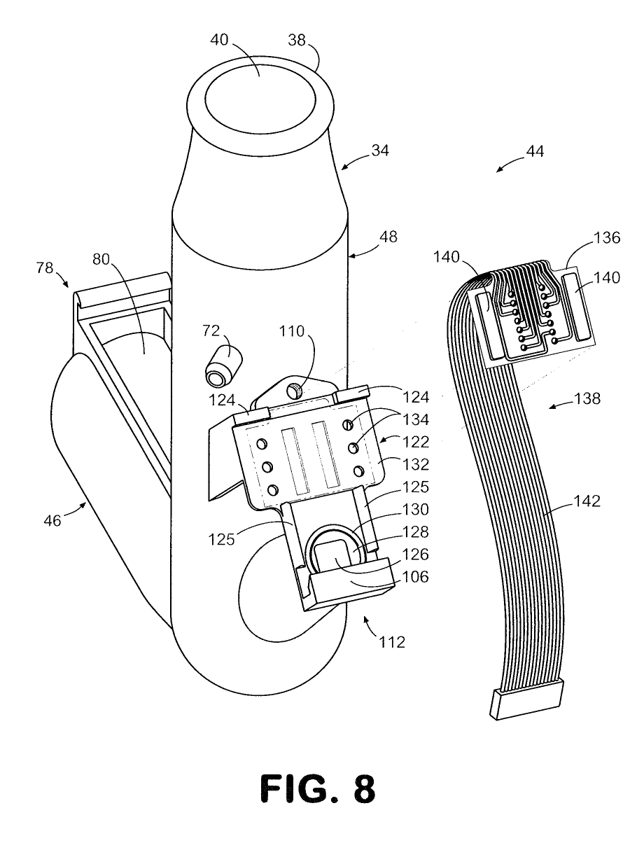

Referring next to FIG. 8, the support structure 112 defines a platform 122 on which the medicine storage and delivery unit 86 is supported. The platform 122 is generally planar and lies within a plane that forms an acute angle with the longitudinal axis of the upper tube 48. Extending upwardly from the platform 122 are upper and lower alignment tabs 124 and 125, respectively, that act as lateral boundary walls for the medicine storage and delivery unit 86. Located near the bottom end of the platform 122 between the lower alignment tabs 125 is an opening 126 that leads to the pathway 120 formed in the medicine injection tube 118 (FIG. 6). In the illustrated embodiment, the opening 126 is generally rectangular (e.g., square) with rounded corners. In some embodiments, the pathway 120 likewise has a generally rectangular (e.g., square) square cross-section with rounded corners. As indicated in FIG. 6, the pathway 120 can be tapered such that it widens as the pathway is traversed from the droplet ejection device 98 to the flow path defined by the drug delivery tube. With further reference to FIG. 8, surrounding the opening 126 is a generally circular recess 128 in which a further sealing member 130, such as an O-ring, is positioned. When provided, the sealing member 130 forms an airtight seal between the droplet ejection device 98 and the pathway 120 and prevents leakage of medicine onto electrical contacts of the droplet ejection device.

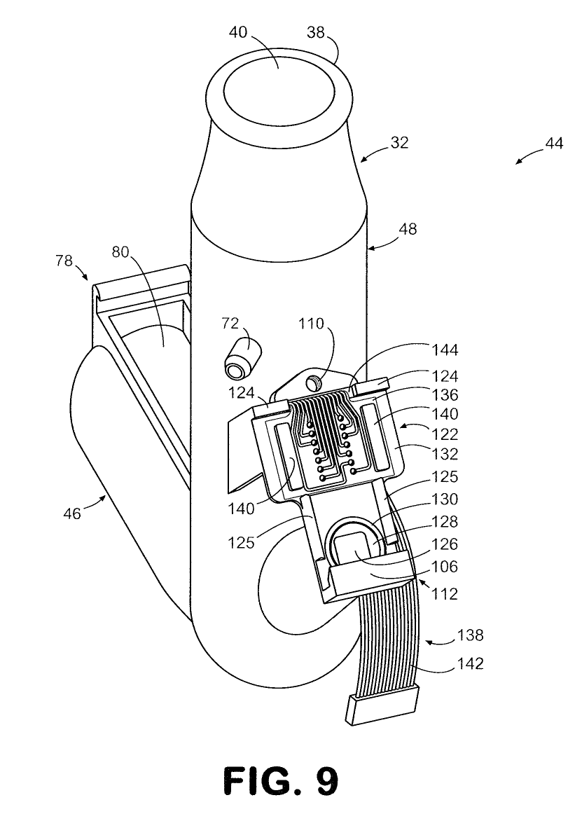

Adjacent the top end of the platform 122 is a seat 132 that is adapted to receive and support a head 136 of an electrical cable 138 that electrically couples the droplet ejection device 98 (FIG. 6) to the circuit board 60 (FIG. 3 and 4). In the illustrated embodiment, the seat 132 comprises various mounting holes 134 that facilitate mounting of the electrical cable head 136 to the platform 122. Exemplary seating of the cable 138 is illustrated in FIG. 9. As indicated in that figure, the head 136 of the cable 138 is positioned on the seat 132 and is secured thereto with attachment elements 140 that extend through the head and into the mounting holes 134. As is further indicated in FIG. 9, the ribbon 142 of the cable 138 extends from the head 136 and through a slot 144 formed in the platform 122 adjacent the opening 110 such that control signals, for example encompassed in a waveform, can be sent from the circuit board 60 to the droplet ejection device 98.

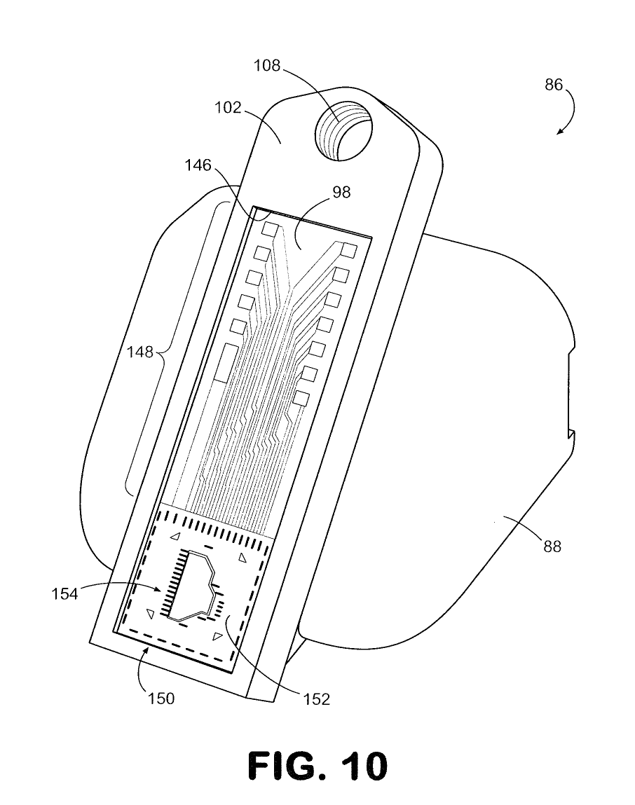

FIG. 10 shows the underside of the medicine storage and delivery unit 86 and the droplet ejection device 98 thereof. As indicated in FIG. 10, the droplet ejection device 98 comprises a rectangular circuit board that is positioned within a rectangular recess 146 formed in the underside of the support member 102. Formed within the droplet ejection device 98 are a plurality of traces 148 that electrically couple an ejection head 150 of the device with contacts provided on the electrical cable head 136. The ejection head 150 comprises a nozzle plate 152 that defines a plurality of nozzles 154 from which droplets of medicine can be ejected. By way of example, the ejection head 150 comprises approximately 5 to 100 such nozzles. Associated with each nozzle 154 is an ejection element (not shown), such as a heater resistor, that, when activated, causes ejection of one of more droplets of medicine. When heater resistors are used, thin layers of medicine within firing chambers (not shown) formed within the ejection head 150 are superheated, causing explosive vaporization and ejection of droplets of medicine through the nozzles 154. Ejection of the droplets creates a capillary action that draws further medicine within the firing chambers such that the droplet ejection device can be repeatedly fired. The size of the droplets depends a least in part on the size of the nozzles 154. In some embodiments, the nozzles 154 are approximately 2 to 1,000 microns (μm) in diameter. In other embodiments, the nozzles 154 are approximately 10 to 400 μm in diameter. In still other embodiments, the nozzles are approximately 75 μm in diameter. Such volumes and sizes can be reproduced with great precision and accuracy with the ejection head 150. Indeed, in regard to precision, testing has confirmed that approximately half of the droplets that are ejected are within approximately 500 nanometers of each other in terms of diameter. In certain embodiments, relatively large nozzles 154, for example on the order of 400 μm in diameter, may be used along with a high duty cycle to provide high mass transfer rates. In such cases, thermal degradation of the ejection head 150 can be avoided or reduced by constructing the ejection head 150 using materials that have high thermal impedance, such as silicon carbide or aluminum nitride deposited on silicon carbide. In some embodiments, the droplets are typically ejected from the nozzles at a velocity of approximately 1 to 7 meters per second (m/s).

Example configurations for the pulmonary drug delivery device 10 having been described in the foregoing, examples of operation of the device will now be described. As explained above, the device 10 can be activated to deliver medicine to the respiratory system of the user upon detecting user inhalation as indicated by a drop in pressure within the upper tube 48 of the drug delivery member 44. The pressure drop can be detected by the pressure sensor 64 and an appropriate detection signal can then be sent from the sensor to the device microcontroller 62. The microcontroller 62 can then activate the fan 50 to cause it to draw in air from the environment, for example through the inlet 28 provided in the front cover 24, and exhaust the air through the opening 80 of the lower tube 46, as indicated by flow arrow 156 in FIG. 6. By way of example, the air is exhausted at a velocity of approximately 0.5 to 3 m/s.

Due to the nature of the fan 50, the air is exhausted at a relatively precise angle relative to the lower tube 46. By way of example, the exhaust angle, α, is approximately 10 to 40 degrees relative to a horizontal direction that is parallel to the longitudinal axis of the lower tube 46. As mentioned above, a sharp angle is formed between the lower tube 46 and the upper tube 48. By way of example, that angle is approximately 70 to 120 degrees, for example approximately 90 degrees. Due to that sharp angle, the air exhausted by the fan 50 impinges upon the walls of the upper tube 48 and becomes highly turbulent within a turbulence zone 158 adjacent the intersection between the lower and upper tubes 46, 48 (i.e., at the sharp “bend” of the drug delivery tube). As is schematically indicated by flow arrows 160, the air vigorously circulates with the turbulence zone 158 before being forced up through the upper tube 48, as indicated by flow arrow 162.

Simultaneous to or soon after activation of the fan 50, the microcontroller 62 activates the droplet ejection device 98 to cause droplets of medicine to be ejected from the nozzles 154 of the ejection head 150. In some embodiments, the nozzles 154 are selectively activated to ensure a desired separation in terms of both distance and time. For example, the nozzles 154 can be activated such that only non-adjacent nozzles eject in sequence and a period of at least approximately 150 to 500 microseconds (μs) passes between firing of any two nozzles. Such an activation scheme ensures that the droplets are physically spaced to a degree at which evaporation of a droplet is not significantly influenced by the proximity of one or more other droplets.

Irrespective of the nozzle activation scheme that is implemented, the ejected droplets travel along the pathway 120 of the medicine injection tube 118 in the direction of arrow 164, which forms an angle, β, of approximately 30 to 60 degrees relative to the horizontal direction and which is generally opposite to the direction of the airflow generated by the fan 50. As indicated in FIG. 6, the droplets are injected directly into the turbulence region 158 so that the droplets enter the airflow within the drug delivery tube at the point of highest turbulence. Injection of the droplets at that site facilitates controlled evaporation that, in turn, shrinks the droplets so that, by the time the droplets reach the opening 40 of the mouthpiece 32, the droplets at or near optimal size for absorption by the lungs.

In order to achieve effective systemic absorption, it is normally desirable to deliver a medicine directly to the alveoli located deep within the lung structure where transport to the bloodstream is most quickly and efficiently accomplished. Lung deposition curves, such as those published by the International Commission on Radiological Protection (ICRP), indicate that the locations within the pulmonary tree in which inhaled particles are deposited depends to a substantial degree upon particle size. Specifically, lung deposition curves based on both theoretical modeling and experimental data typically show that particle deposition rates in the alveolar regions of the lung are greatest for particles having a diameter of approximately 1 to 3 μm. In view of this, the device 10 can be configured to deliver droplets having a diameter of approximately 1 to 3 μm from the opening 40 of the mouthpiece 34. In other embodiments, the droplets have even smaller exit diameters, for example approximately 0.1 to 1 μm, to enable hygroscopic growth of the droplets within the respiratory tract.

With further reference to FIG. 6, it is noted that the droplets exit the droplet injection tube 118 at a location and angle at which the droplets must travel a relatively long distance before reaching the walls of the lower tube 46. In addition to increasing flight time, which further assists in the evaporation process, such an arrangement reduces the likelihood that the droplets will be deposited upon the tube walls instead of being delivered to the user's respiratory tract.

After the desired quantity of medicine has been injected into the airflow during the current inhalation cycle, ejection of medicine droplets ceases and the fan 50 is powered down. The process can then be repeated for further inhalation cycles of the user until a desired dosage of medicine has been administered. If desired, the entire process can be repeated at a later time, such as later that day or the next day. In some embodiments, appropriate controls can be integrated into the device 10 to limit the frequency with which the medicine can be administered. For example, the microcontroller 62 can be programmed to limit operation of the device 10 once every hour, once every few hours, once each day, and the like.

As mentioned above, it may be desirable to deliver droplets having a diameter of approximately 1 to 3 μm from the opening 40 of the mouthpiece 34. It is possible, however, for the size of the droplets to fall outside of that range in some circumstances. For example, environmental conditions, such as temperature, humidity, and pressure, can cause the ejected droplets to shrink or grow. In some embodiments, measures may be taken, substantially in real time, to control the size of the droplets relative to feedback that is collected by the device 10. Such feedback can comprise, for example, one or more of the current atmospheric temperature, humidity, and pressure, or the size of the droplets that are being delivered. In the former case, the device 10 comprises an open feedback loop and, in the latter case, the device comprises a closed feedback loop. Irrespective of which feedback scheme is used, the actions to be taken can be determined through reference to a look-up table or through application of an appropriate algorithm, either of which can be stored within memory provided on the circuit board 60.

Generally speaking, the size of the droplets can be controlled before droplet ejection, during droplet ejection, and after droplet ejection. Various embodiments for controlling droplet size before, during, and after ejection are described in the following.

Prior to droplet ejection, the temperature of the medicine to be administered can be adjusted to control the size of the droplets that will be ejected. For example, relatively smaller droplets can be generated when the medicine is heated given that elevated temperatures decrease both the viscosity and surface tension of liquids, which translates into smaller droplets being ejected. In some embodiments, liquid temperatures in the range of approximately 45 to 110° C. are effective in reducing droplet diameter, with temperatures of approximately 90 to 99° C. being preferred in some embodiments.

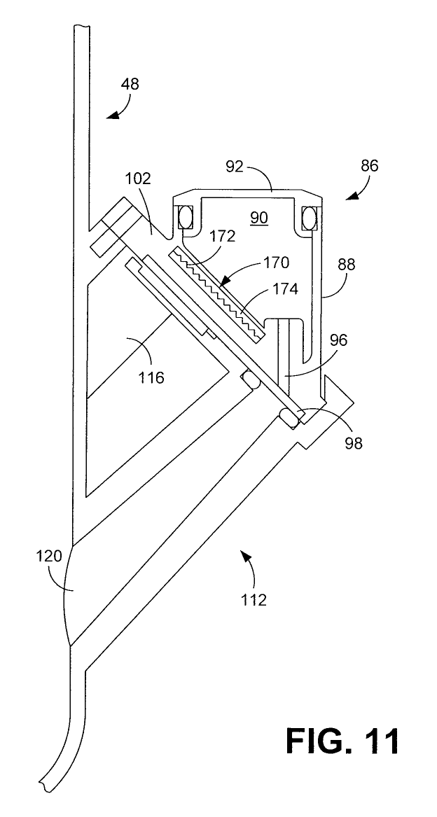

Medication used in the device 10 can be heated using a variety of methods. Generally speaking, any method with which the medication is heated prior to its ejection (i.e., is preheated) can be used. FIG. 11 illustrates a first preheating implementation. As indicated in FIG. 11, the medicine storage and delivery unit 86 includes a heating element 170 provided within the support member 102 and placed in close proximity to the interior space 90 that contains the medicine to be administered. By way of example, the heating element 170 comprises a resistance heater that includes a heating coil 172 that is contained or encapsulated within a thermally-conductive member 174.

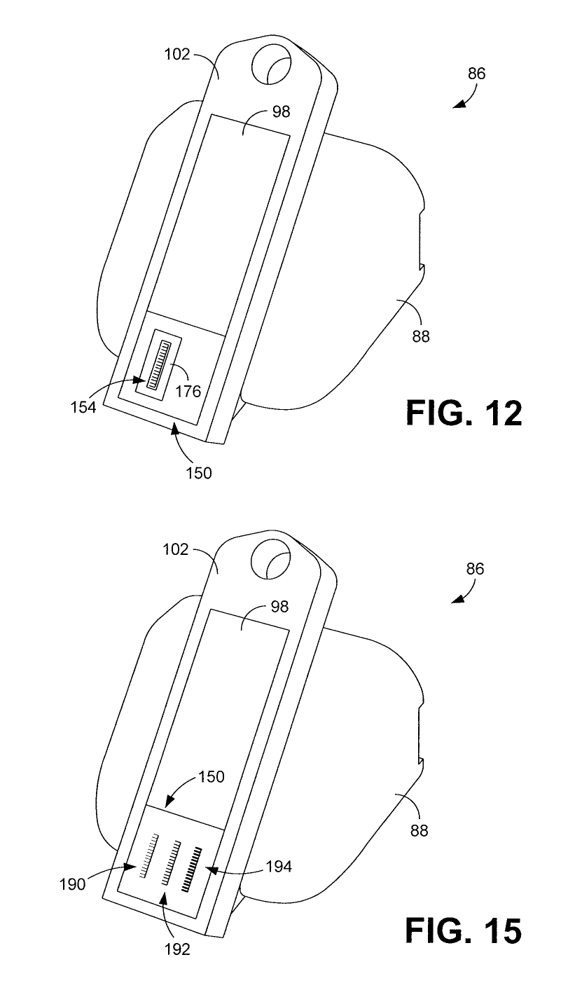

FIG. 12 illustrates a second preheating implementation. As indicated in FIG. 12, the ejection head 150 includes a heating element 176 that is provided in close proximity to the nozzles 154. By way of example, the heating element 176 comprises a resistive element that surrounds the nozzles and provides resistance to an applied voltage.

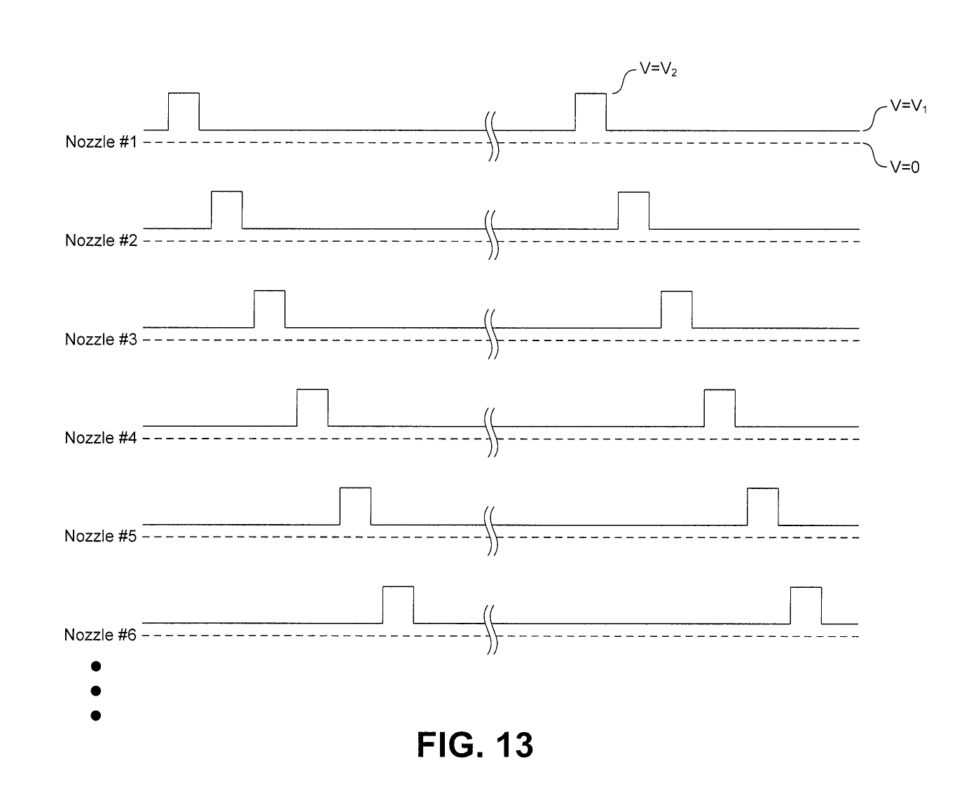

In a further embodiment, preheating can be achieved using the ejection elements of the droplet ejection head 150. For example, when the ejection elements comprise heater resistors, a relatively low voltage can be applied to the resistors when they are not being used to eject droplets so as to heat the medicine prior to such ejection. FIG. 13 illustrates such control with a timing diagram that illustrates the timing for pulses that are applied to ejection elements associated with various nozzles (i.e., nozzles 1-6). Although six nozzles are identified in FIG. 13 to facilitate this discussion, a greater or lesser number of nozzles, and therefore ejection elements, can be used.

As indicated in FIG. 13, the heater resistors associated with the nozzles are sequentially pulsed without overlap. When a pulse is applied, a relatively high voltage, V₂, is applied to the heater resistor to superheat the medicine and eject droplets. Instead of reducing the voltage applied to the heater resistors to zero after a pulse, however, a relatively low voltage, V₁, is still applied to the heater resistor so as to heat the medicine that replaces the medicine that was just ejected by the heater resistor.

In a variation on the control scheme described above in relation to FIG. 13, one or more heater resistors of the ejection head 150 can be utilized as designated preheaters that are not used to eject droplets. For example, alternate heater resistors within an aligned row can be used to preheat medicine that is to be ejected by adjacent heater resistors.

Yet another parameter that has a significant effect on the size of the droplets that are ejected is the composition of the medicine. In particular, the nature of the medicine used to form the droplets can have a significant effect on the rate at which the droplets evaporate. The evaporation rate of droplets depends to a significant degree on the properties of the solvent and the solutes present within the solvent. Volatile liquids (i.e., those with relatively high vapor pressures) evaporate more quickly than non-volatile liquids. Various solutes tend to affect the vapor pressure of the droplet surface in particular ways. Saline solutions, which comprise water and sodium chloride, are widely used as carriers for medicinal compounds due to their similarity to and compatibility with human tissues and biological processes. The presence of sodium chloride in such solutions tends to lower vapor pressures.

Evaporation and condensation typically occur simultaneously at the air-liquid interface of liquid droplets. The ratio of evaporation rate to condensation rate is dependent upon the vapor pressure at the droplet surface. As the concentration of sodium chloride in a saline solution increases, the ratio of evaporation to condensation decreases. At low relative humidity and elevated temperatures, saline solutions (e.g., a 0.9% solution) tend to have evaporation rates that are higher than condensation rates with a net result of evaporation and droplet shrinkage. As relative humidity increases, the rate of condensation relative to evaporation becomes larger until the droplet begins to gain mass and increase in size. Increasing the solute concentration in such a case will shift the point at which evaporation and condensation are at equilibrium to a point of lower humidity and higher temperature.

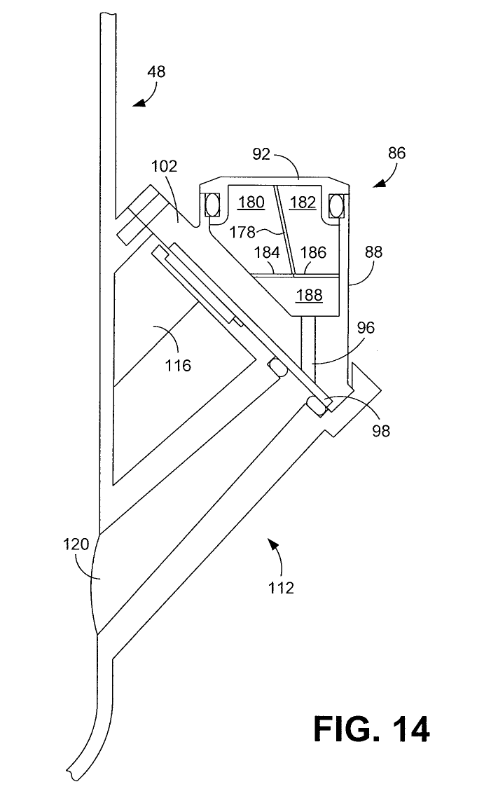

FIG. 14 illustrates an embodiment in which the composition of the administered medicine can be altered prior to ejection. In the embodiment, a medicine storage and delivery unit 86 has a medicine container 88 that includes a divider wall 178 that defines two separate compartments 180 and 182 in which two different compounds (e.g., liquids) can be stored. In addition, the unit 86 includes control elements 184 and 186 that are used to control the relative amounts of each compound that are delivered from the compartments 180, 182 to a mixing chamber 188 in which the compounds can be mixed prior to ejection. By way of example, the control elements 184,186 can comprise further droplet ejection devices or other liquid metering devices. After the compounds are mixed, the resulting solution can be delivered to the droplet ejection device 98 along the passage 96. Optionally, a mixing device, such as a mechanical agitator (not shown), can be used within the chamber 188 to ensure adequate mixing before ejection.

When separate compartments 180, 182 are used as described above, the composition of the medicine that is ejected can be controlled. For example, the first compartment 180 can contain a concentrated medicine while the second compartment 182 can contain an inert liquid, such as saline solution. The amounts of liquid that are provided into the mixing chamber 188 from each compartment 180, 180 can be controlled with the control elements 184, 186 relative to measured feedback as to environmental conditions and/or droplet sizes.

As mentioned above, certain parameters affect droplet size at the time of ejection. One such parameter is the size of the nozzles that are used to eject the droplets, which directly affects the size of the droplets. Such nozzle size variability can be achieved by providing a droplet ejection device that comprises nozzles of various different sizes. FIG. 15 illustrates an example of such an arrangement. In particular, FIG. 15 illustrates the medicine storage and delivery unit 86 including a droplet ejection device 98 having an ejection head 150 that is provided with three rows of nozzles having different sizes. In particular, a first row 190 comprises small-sized nozzles, a second row 192 comprises medium-sized nozzles, and a third row 194 comprises large-sized nozzles. With such an arrangement, the nozzles that are used to eject droplets can be selected based upon feedback that is received. For example, if the medium-sized nozzles are being used and it is determined that the droplets are too small (e.g., due to humid environmental conditions), control can be exercised over the droplet ejection device 98 to switch to ejection using the large-sized nozzles.

As also mentioned above, the size of the droplets can be controlled after ejection. Therefore, even if the droplets entering the drug delivery member 48 are undesirably small or large, their size can be adjusted to ensure that the droplets enter the user's mouth at the optimal size (e.g., approximately 1 to 3 μm). The exercise of such control may generally be referred to as post-processing of the droplets.

It has been determined that droplet size can be significantly reduced due to evaporation of the ejected droplets during their flight to the user's respiratory tract. Such evaporation may naturally occur as a consequence of the current environmental conditions in which the system is used, such as temperature, humidity, and pressure. As the droplets evaporate, they lose fluid (e.g., water), which results in a corresponding loss of mass and volume and, ultimately, droplet diameter. Discussed in the following are several parameters that affect droplet evaporation rate and which therefore can be used to control droplet size.

One parameter that has a significant impact on droplet evaporation is air temperature. Specifically, the higher the temperature of the air that is being used to deliver the droplets to the respiratory tract, the greater the evaporation rate. Therefore, droplet size can be reduced by heating the air that flows through the system. In some embodiments, the air is heated from an ambient temperature (e.g., room temperature) to a temperature of approximately 20 to 60° C. The extent of droplet evaporation and size reduction obtained is dependent upon the particular air temperature that is reached as well as the duration of time the droplets are present within the heated air (i.e., time of flight to the respiratory tract), with higher temperatures and longer times of flight resulting in greater evaporation. The time of flight corresponds to the distance the droplets must travel to reach the respiratory tract and the speed with which the air is flowing toward the user. Therefore, the temperature to which the air is heated, the position at which the drug delivery unit is located relative to the patient interface, and the speed setting for the air supply blower can each be selected to obtain desired evaporation results.

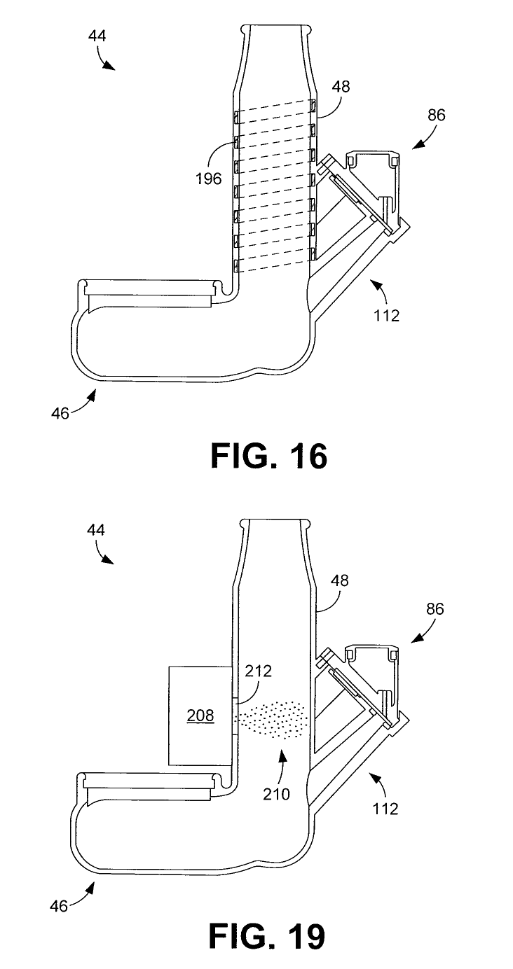

FIG. 16 illustrates an embodiment in which the upper tube 48 of the drug delivery member 44 comprises a heating element 196. In this embodiment, the heating element 196 comprises a resistive coil that is integrated into the walls of the upper tube 48. Although the heating element 196 is shown in FIG. 16 as being provided within the walls of the upper tube 48, the heating element alternatively could be provided within the flow path defined by the tube, if desired.

In a further embodiment, ejected droplets can be heated by exposing the droplets to electromagnetic radiation. Such exposure can result in rapid temperature increase and, consequently, evaporation of the droplet. Generally speaking, substances absorb electromagnetic energy to different degrees depending on the wavelengths of the energy that is applied with the greatest overall absorption for a given liquid being achievable by selecting a wavelength that offers the greatest absorption for that liquid. By calculating the volume of the droplet and then using the heat of vaporization for the liquid of interest, the amount of energy required to evaporate the droplet can be determined

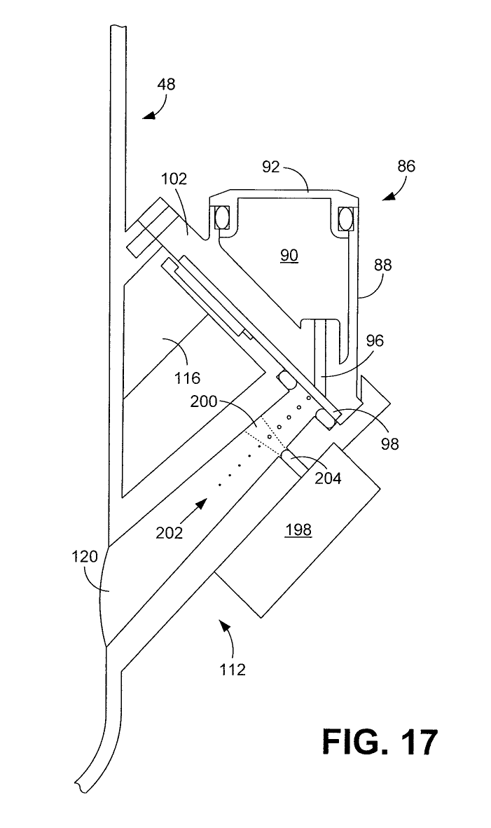

FIG. 17 illustrates a first embodiment in which electromagnetic energy is used to control (i.e., reduce) droplet size. In FIG. 17, the medicine storage and delivery unit 86 is provided with an electromagnetic energy source 198 that is used to generate electromagnetic energy 200 through which ejected droplets 202 travelling along the passage 120 pass. By way of example, the electromagnetic energy 200 comprises laser light emitted by a laser diode 204 having a central wavelength of approximately 2,700 nanometers. In some embodiments, the laser diode 204 can comprise a 15 milliwatt (mW) laser diode that emits light from a “window” having an area of about 100 square microns. This relates an energy intensity of about 15,000 watts per square centimeter (w/cm²).

As a consequence of the droplets 202 passing through the electromagnetic energy 200, the droplets are rapidly heated and therefore rapidly evaporated so as to shrink. Such shrinkage is depicted in FIG. 17 with different sized droplets of exaggerated scale.

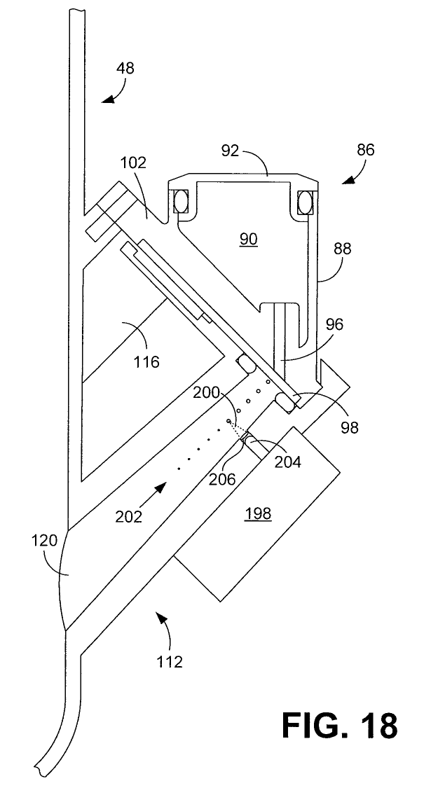

FIG. 18 illustrates an alternative embodiment in which electromagnetic energy is used to control (i.e., reduce) droplet size. In FIG. 18, the medicine storage and delivery unit 86 is provided with an electromagnetic energy source 198 similar to that described above in relation to FIG. 17. In the embodiment of FIG. 18, however, one or more focusing lenses 206 is/are used to focus the light emitted from the laser diode 204 to concentrate the electromagnetic energy on individual droplets 202.

In either of the embodiments of FIGS. 17 and 18, the electromagnetic energy source 198 can either operate continuously during a given period of droplet ejection or can be intermittently fired to target individual droplets. In the latter case, the duration of operation of the electromagnetic energy source 198 is reduced, thereby reducing energy consumption. When the electromagnetic energy source 198 is intermittently fired to intercept individual droplets, the source and its diode 204 are controlled in relation to the timing of droplet ejection by the droplet ejection device 98. In other words, diode firing is coordinated with ejection element firing, taking into account the distance between the ejection elements and the diode and therefore an appropriate time delay. If, desired, the electromagnetic energy source 198 and its diode 204 can be controlled to apply different amounts of energy into individual droplets by varying the intensity of the diode output. Therefore, an even greater amount of control can be exercised over droplet size.

Although a laser diode has been explicitly identified above, it is noted that other electromagnetic energy sources may be used with desirable results. For example, in some embodiments, light emitting diodes (LEDs) can be used in place of laser diodes.

Another parameter that has a significant effect on droplet size is the relative humidity of the air used to carry the droplets to the user. As one would expect, the lower the relative humidity of the air, the greater the droplet evaporation rate and therefore the smaller the diameter of the droplets when they reach the respiratory tract. In some embodiments, the air is dehumidified from an initial relative humidity to a reduced relative humidity. The extent of droplet evaporation and size reduction that can be achieved is dependent upon the particular environmental relative humidity and the duration of time the droplets are present within the airstream (time of flight), which corresponds to both the distance the droplets must travel to reach the respiratory tract and the speed with which the air that carries the droplets is flowing. Therefore, the relative humidity to which the air is reduced, the position at which the drug delivery unit is located relative to the patient interface, and the speed setting for the air supply blower can each be selected to obtain desired evaporation results. Just as dehumidification may be used as a means to decrease the size of the medicine droplets, humidification may be used to increase the size of the medicine droplets.

FIG. 19 illustrates an embodiment of a drug delivery member 44 that includes a conditioning unit 208 that can be used to reduce and/or increase the relative humidity of air expelled by the unit's blower. In terms of humidification, the conditioning unit 208 can comprise one or more of a vaporizer, nebulizer, or other atomizer configured to vaporize a liquid (e.g., water) for provision into the flow path of the drug delivery tube 44. In other embodiments, humidification can be provided with a droplet ejection device mechanism similar to that used to administer the medicine. Regardless, the generated vapor 210 can be provided into the flow path via a passage 212, for example provided within the upper tube 48 of the drug delivery member 44. In terms of dehumidification, the conditioning unit 208 can comprise one or more of desiccant material and a condenser that removes moisture from the flow path and therefore the ejected droplets.

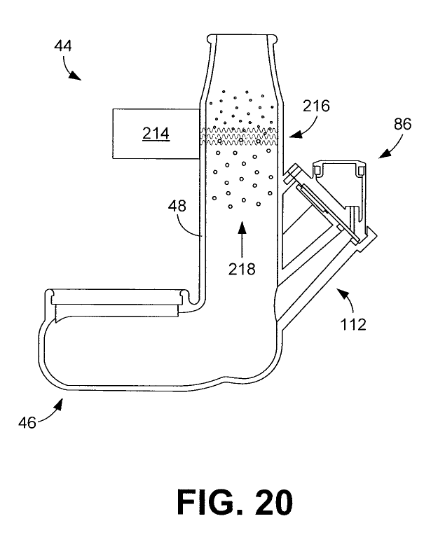

Another method with which droplet size can be controlled, and more particularly reduced, is to break up relatively large droplets into smaller droplets as they travel along the flow path of the drug delivery device. FIG. 20 depicts such an action. As indicated in FIG. 20, the drug delivery member 44 is provided with a sonic wave generator 214 with which sonic waves 216, such as ultrasonic waves, can be created and applied to droplets traveling through the member. When generated, such waves 216 can form a field through which droplets 218 pass as they are carried toward the outlet of the drug delivery member 44. As the droplets 218 pass through the field, the waves 216 break up the droplets into smaller droplets, as is depicted in FIG. 20 with exaggerated scale.

In the foregoing, various parameters have been described that affect droplet evaporation and that therefore can be manipulated to control droplet size. Although each parameter is discussed separately, two or more of the parameters can be individually or simultaneously controlled in order to achieve a desired droplet size.

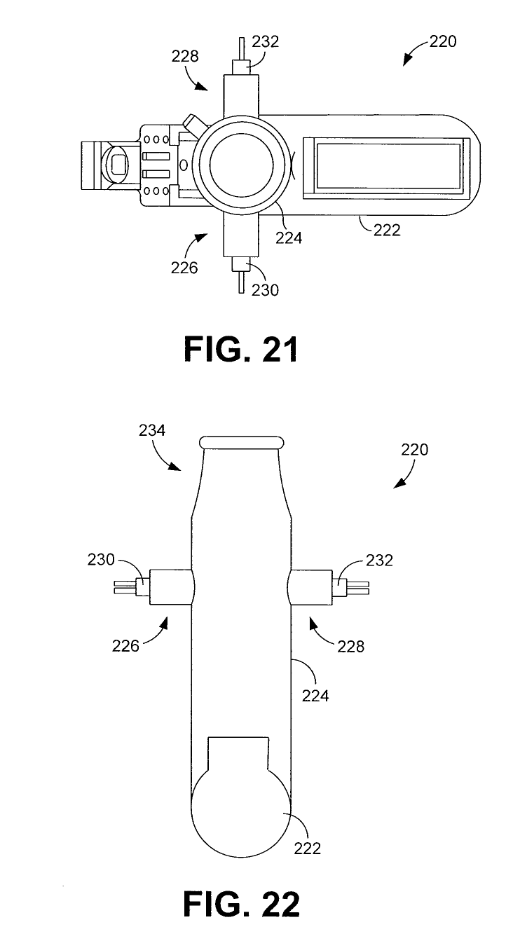

In cases in which the current atmospheric temperature, humidity, and pressure are to be measured (i.e., open-loop feedback), the circuit board 60 can, as described above, include one or more sensors 65 for detecting those conditions (see FIG. 4). In cases in which the size of the droplets is to be measured (i.e., closed-loop feedback), the device 10 can comprise appropriate droplet size sensing apparatus. FIGS. 21 and 22 illustrate an alternative drug delivery member 220 that includes such apparatus. As indicated in those figures, the drug delivery member 220 is similar in many ways to the drug delivery member 44. Therefore, the drug delivery member 220 comprises a first or lower tube 222 in communication with a second or upper tube 224. Provided on the upper tube 224, however, are two ports 226 and 228 that provide access to the interior of the upper tube. Associated with the first port 226 is a light source 230 and associated with the second port 228 is a light detector 232. By way of example, the light source 230 comprises an LED that emits laser light toward the light detector 232, which can comprise a photoelectric sensor.

The light source 230 and light detector 232 together comprise a droplet size sensing apparatus configured to capture light data regarding the droplets flowing through the upper tube 224. As indicated in FIG. 22, both the light source 230 and the light detector 232 can be located at a position near the end of the upper tube 224 adjacent the mouthpiece 234 so as to provide data relevant to the size of the droplets just before they exit the mouthpiece and enter the patient. Accordingly, the approximate size of the droplets being administered can be determined and adjustments can be made to modify the sizes of later-ejected droplets, if necessary.

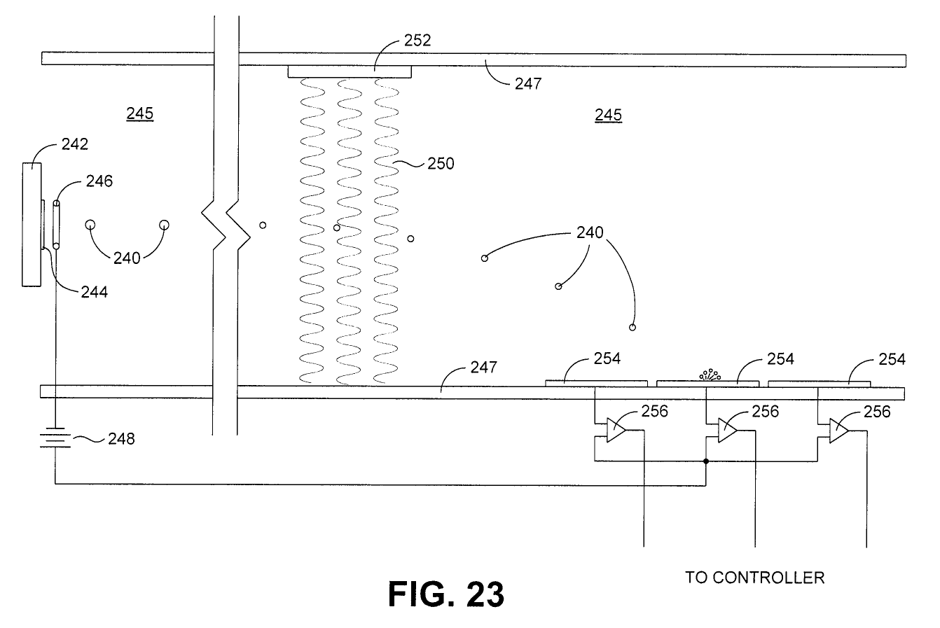

Droplet size can be measured in alternative ways. In one such alternative, ejected droplets are electrically charged and passed through an electric or magnetic field that laterally (relative to the direction of flight) deflects the droplets. When the resultant degree of deflection is determined, the mass, and therefore the size, of the droplets can be inferred. FIG. 23 illustrates apparatus that can be used in such a process.

As shown in FIG. 23, droplets 240 are ejected from a droplet ejection device 242 through a metal nozzle plate 244 into a flow path 245 bounded by one or more walls 247 (e.g., of the drug delivery tube). An electrode 246 is positioned in close proximity to the nozzle plate 244 from which the droplets 240 are ejected. In some embodiments, the electrode 246 is formed as a ring through which the droplets 240 pass. Regardless, the electrode 246 is held at a high positive electrical potential by a power source 248, such as a battery. The potential on the electrode 246 draws electrons to the nozzle plate 244 and onto the droplets 240 as they are formed, thereby providing them with a net negative charge. Although, the droplets 240 may experience significant evaporation soon after being ejected from the droplet ejection device 242, the net charges on the droplets 240 will change very little.

Later along the flow path 245, the droplets 240 pass through an electric or magnetic field 250 generated by a field generator 252. In some embodiments, the field generator 252 comprises one or more permanent magnets or electromagnets. In other embodiments, the field generator 252 comprises opposed plates provided on opposite sides of the flow path 245 (not shown) having a large potential difference. For an electric field, a force is imposed upon the droplets 240 given by the following relation:

F=qE [Equation 1]

where q is the charge on the droplets and E is the strength of the electric field. For a magnetic field, a force is imposed upon the droplets 240 given by the following relation:

F=qvβsinθ [Equation 2]

where q is the charge on the droplet, v is the velocity of the droplet, β is the strength of the magnetic field, and θ is the angle between the direction of travel and the magnetic field. The direction of the force, F, whether due to an electric or magnetic field, is perpendicular to direction of travel of the droplet 240. In FIG. 23, the droplets 240 are traveling from left to right along the flow path 245. Therefore, the droplets 240 are laterally deflected toward a wall 247 that bounds the flow path 245 (i.e., downward in FIG. 23).

Because the charge on the droplets 240 remains substantially constant as evaporation occurs, the droplets enter the field 250 with nearly identical charges. Therefore, the lateral force imposed on the droplets 240 is substantially constant. However, the masses of the droplets will differ depending upon how much evaporation has occurred. It follows then that the smaller the droplet 240, the greater the force will affect the droplet and the greater the degree of deflection. Therefore, the size of the droplet 240 can be inferred from the amount of deflection of the droplet.

The deflection of the droplets 240 can be determined using conductive pads 254 placed on the wall 247. As indicated in FIG. 23, the pads 254 are linearly spaced along the flow path 245 so that the pad a given droplet 240 strikes provides an indication of the degree of droplet deflection by the field 250. Each pad 254 can be individually monitored with an amplifier circuit, such as a field effect transistor (FET) circuit, that includes an amplifier 256. When a droplet 240 strikes a pad 254 as shown in FIG. 23 (middle pad 254), the charge on the droplet is transferred to the pad, thereby creating a small electrical signal that can be amplified by the amplifier circuit and supplied as a feedback signal to a controller. Accordingly, the output from the amplifier circuits can be used to determine where the droplets land, the degree of deflection of the droplets, the droplet mass, and therefore the droplet size (e.g., diameter).

Notably, if the amplifier circuits are highly sensitive, they further can be used to detect passing droplets. In such a case, it would be possible to measure the speed of the droplet by determining the times at which the droplets pass the various contact pads. It is further noted that the applied charges can, in some embodiments, be used to adjust the size of the particles. Once a droplet is charged, the excess electrons arrange themselves on the surface of the droplet. Having like charges, the electrons naturally repel each other. As the droplet evaporates and the electrons are forced closer together, the repulsive forces increase. If the repulsive forces become great enough, they may break the droplet apart into multiple smaller droplets. Therefore, at a given charge level and percentage evaporation, droplet charge create smaller particles.

Various modifications can be made to the embodiments described in the foregoing. For example, in one alternative embodiment, an extension tube can be connected to the mouthpiece of the device and used to increase the distance between the device housing and the point at which medicine enters the user's mouth. In another alternative embodiment, the cap to the medicine container can include a vent port that equalizes the pressure within the container with that of the surrounding environment. In a further alternative embodiment, a screen can be placed over the passage formed within the container to filter particulate matter that could clog the droplet ejection device and/or to reduce surface tension that could interfere with the flow of medicine to the drug ejection device.

It is further noted that appropriate regulatory measures can be taken to avoid abuse of the device or the medicine(s) that the device is intended to administer. For example, each medicine storage and delivery unit can be sold separately as one-time use component that comprises identification data that can be read by the pulmonary drug delivery device when the unit is installed on the drug delivery member. If the device microcontroller determines from the identification data that the medicine storage and delivery unit does not contain a medicine for which the device has been prescribed, for example by a doctor, operation of the device can be disabled.

Finally, it is noted that absolute spatial terms such as “horizontal” and “vertical” have been used herein relative to the orientations of the device components shown in the drawings. Therefore, it is to be understood that such terms may not strictly apply in cases in which the orientation of the device is changed from that shown in the figures.

The following are claimed:

1. A pulmonary drug delivery device comprising:
a drug delivery tube that defines a flow path;
a droplet ejection device configured to eject droplets of medication into the flow path; and
a medicine heating device configured to heat the medicine before it is ejected by the droplet ejection device.

2. The pulmonary drug delivery device of claim 1, wherein the medicine heating device comprises a heating element provided in close proximity to a container of the pulmonary drug delivery device in which the medicine is held prior to ejection.

3. The pulmonary drug delivery device of claim 2, wherein the heating element is a resistance heater provided within a medicine storage and delivery unit of the pulmonary drug delivery device.

4. The pulmonary drug delivery device of claim 1, wherein the medicine heating device comprises a heating element that is provided in close proximity to nozzles of the droplet ejection device.

5. The pulmonary drug delivery device of claim 4, wherein the heating element comprises a resistive element provided on a nozzle plate of the droplet ejection device.

6. The pulmonary drug delivery device of claim 1, wherein the medicine heating device comprises a heater resistor of the droplet ejection device used to eject droplets to which a relatively low voltage is applied, the relatively low voltage being too low to heat the heater resistor to a point at which it will eject a droplet but high enough to heat medicine contained in a firing chamber associated with the heater resistor.

7. A pulmonary drug delivery device comprising:
a drug delivery tube that defines a flow path;
a droplet ejection device configured to eject droplets of medication into the flow path; and
a medicine container in which medicine is held prior to ejection by the droplet ejection device, the medicine container including separate compartments in which different compounds can be stored and a mixing chamber in which desired amounts of each compound can be mixed prior to delivery to the droplet ejection device.

8. The pulmonary drug delivery device of claim 7, wherein the medicine container comprises a divider wall that separates the compartments and control elements that are used to control relative amounts of each compound that are delivered to the mixing chamber for mixing.

9. The pulmonary drug delivery device of claim 7, further comprising a mixing device provided within the mixing chamber that mixes the compounds together.

10. A pulmonary drug delivery device comprising:
a drug delivery tube that defines a flow path; and
a droplet ejection device configured to eject droplets of medication into the flow path, the droplet ejection device having an ejection head that includes ejection nozzles having different sizes, the different sized ejection nozzles being alternatively selectable to control the size of droplets that are ejected from the droplet ejection device.

11. The pulmonary drug delivery device of claim 10, wherein the ejection head comprises small-sized nozzles and large-sized nozzles.

12. The pulmonary drug delivery device of claim 11, wherein the ejection head further comprises medium-sized nozzles.

13. The pulmonary drug delivery device of claim 10, wherein the different sized ejection nozzles are arranged in separate rows on the ejection head.

14. A pulmonary drug delivery device comprising:
a drug delivery tube that defines a flow path;
a droplet ejection device configured to eject droplets of medication into the flow path; and
a droplet heating device configured to heat the droplets of medicine that have been ejected by the droplet ejection device.

15. The pulmonary drug delivery device of claim 14, wherein the droplet heating device comprises a heating element associated with the drug delivery tube.

16. The pulmonary drug delivery device of claim 15, wherein the heating element comprises a resistive coil associated with walls of the drug delivery tube.

17. The pulmonary drug delivery device of claim 16, wherein the resistive coil is integrated into the walls of the drug delivery tube.

18. The pulmonary drug delivery device of claim 14, wherein the droplet heating device comprises an electromagnetic energy source that emits electromagnetic energy through which ejected droplets pass.

19. The pulmonary drug delivery device of claim 18, wherein the electromagnetic energy source comprises a laser.

20. The pulmonary drug delivery device of claim 18, wherein the electromagnetic energy source includes one or more focusing lenses that focus the electromagnetic energy on individual droplets.

21. A pulmonary drug delivery device comprising:
a drug delivery tube that defines a flow path;
a droplet ejection device configured to eject droplets of medication into the flow path; and
a conditioning unit configured to control the humidity of air within the drug delivery tube.

22. The pulmonary drug delivery device of claim 21, wherein the conditioning unit comprises a device configured to vaporize a liquid for provision into the flow path.

23. The pulmonary drug delivery device of claim 21, wherein the conditioning unit comprises a desiccant material or a condenser that removes humidity from the air.

24. A pulmonary drug delivery device comprising:
a drug delivery tube that defines a flow path;
a droplet ejection device configured to eject droplets of medication into the flow path; and
a droplet size control device that breaks apart ejected droplets into smaller sized droplets.

25. The pulmonary drug delivery device of claim 24, wherein the droplet size control device comprises a sonic wave generator that generates sonic waves through which the ejected droplets pass.

26. The pulmonary drug delivery device of claim 24, wherein the droplet size control device comprises an ultrasonic wave generator that generates ultrasonic waves through which the ejected droplets pass.

27. A pulmonary drug delivery device comprising:
a drug delivery tube that defines a flow path;
a droplet ejection device configured to eject droplets of medication into the flow path;
a controller that controls operation of the droplet ejection device; and
a feedback system configured to provide feedback to the controller indicative of the size of droplets that are being administered to a user to enable the controller to take action to adjust the size of the droplets.

28. The pulmonary drug delivery device of claim 27, wherein the feedback system comprises a sensor that measures a condition that affects droplet size.

29. The pulmonary drug delivery device of claim 28, wherein the sensor comprises a temperature sensor.

30. The pulmonary drug delivery device of claim 28, wherein the sensor comprises a humidity sensor.

31. The pulmonary drug delivery device of claim 28, wherein the sensor comprises a pressure sensor.

32. The pulmonary drug delivery device of claim 27, wherein the feedback system comprises droplet size sensing apparatus.

33. The pulmonary drug delivery device of claim 32, wherein the droplet size sensing apparatus comprises a light source configured to shine light on ejected droplets and a light detector configured to capture light data regarding the droplets.

34. The pulmonary drug delivery device of claim 33, wherein the light source comprises a laser light emitting diode (LED) and the light detector comprises a photoelectric sensor.

35. The pulmonary drug delivery device of claim 32, wherein the droplet size sensing apparatus comprises an electrode that draws electrons to droplets ejected by the droplet ejection device to provide the droplets with a negative charge and a field generator that generates a field through which the negatively-charged droplets later pass, the field imposing a force on the droplets.

36. The pulmonary drug delivery device of claim 35, further comprising contact pads arranged in different positions along a length of the flow path that detect contact of the charged droplets after they have been deflected by the imposed force, wherein the position of the contact pad that receives a given droplet provides an indication of the amount of droplet deflection and therefore droplet size.

37. A method for controlling the size of droplets of medicine that are administered to a user, the method comprising:
ejecting droplets of medicine with a droplet ejection device of a pulmonary drug delivery device;
obtaining feedback relevant to the size of the droplets that are being administered to the user; and
taking action to adjust the size of the droplets.

38. The method of claim 37, wherein taking action comprises heating the medicine before it is ejected.

39. The method of claim 37, wherein taking action comprises controlling a composition of the medicine before it is ejected.

40. The method of claim 37, wherein taking action comprises selecting of a particular size of nozzle of the droplet ejection device to use to eject the droplets.

41. The method of claim 37, wherein taking action comprises heating air that carries the droplets to the user.

42. The method of claim 37, wherein taking action comprises changing the humidity of air that carries the droplets to the user.

43. The method of claim 37, wherein taking action comprises heating the ejected droplets with electromagnetic energy.

44. The method of claim 37, wherein taking action comprises breaking up the ejected droplets into smaller sized droplets with sonic waves.

45. The method of claim 37, wherein obtaining feedback comprises measuring a temperature.

46. The method of claim 37, wherein obtaining feedback comprises measuring a humidity.

47. The method of claim 37, wherein obtaining feedback comprises measuring a pressure.

48. The method of claim 37, wherein obtaining feedback comprises applying deflective force to the ejected droplets and determining an extent to which the droplets are deflected, that extent being indicative of the size of the droplets.

49. The method of claim 48, wherein applying deflective force comprises applying a negative charge to the droplets and passing the droplets through an electrical or magnetic field.
